(19) United States
(12) Patent Application Publication
(10) Pub. No.: US 2012/0252088 A1
(43) Pub. Date: Oct. 4, 2012

(54) FLOATING MAGNET PROBE FOR CELL ISOLATION

(76) Inventors: Jie Zhang, San Jose, CA (US); Glenn Y. Deng, San Jose, CA (US); Fang Tien, Palo Alto, CA (US)

(21) Appl. No.: 13/296,817

(22) Filed: Nov. 15, 2011

Related U.S. Application Data
(60) Provisional application No. 61/468,668, filed on Mar. 29, 2011.

(51) Int. Cl. C12M 1/42; C12N 13/00

(52) U.S. Cl. 435/173.9; 435/283.1

(57) ABSTRACT

A cell isolation device with an inner freely floating magnetic probe to capture and collect biological cells, such as circulating tumor cells (CTCs), during immunomagnetic incubation provides a highly efficient, low cost method to collect target cells at a single cell level. A special membrane filter may be placed above a wash well to enable separation of collected cells from free magnetic beads to yield high purity cells enabling accurate cell count. The novel approach provides a faster turn-around to separate target cells from blood, marrow or other samples with efficient removal of free magnetic beads. Cancel cell spiking experiments show a recovery rate of greater than 85%.

### CROSS REFERENCE OF RELATED APPLICATIONS

The present application claims the benefit of U.S. Provisional Application No. 61/468,668, filed on Mar. 29, 2011 for “Floating Magnet Probe for Cell Isolation”, which is hereby incorporated by reference in its entirety.

### STATEMENT REGARDING FEDERALLY SPONSORED RESEARCH OR DEVELOPMENT

None

### BACKGROUND OF THE INVENTIONS

1. Field of Invention

This invention relates generally to cell isolation devices, and more specifically, a device with a floating magnetic probe used to isolate biological cells.

2. Background

Cell isolation devices are very helpful and sometimes even necessary in many areas, including, but are not limited to, life sciences research, healthcare study, and medical treatment. An isolated cell, after enrichment, isolation, and purification, can be used in subsequent downstream tests and measurements, such as analyzing DNA mutation and RNA/protein expression at single cell level, understanding certain tumor formation mechanism and metastatic processes, detecting or monitoring various deceases, performing pathology analysis, documenting a person's identity, and classifying animal species.

Certain decease tends to create specific type of cells (or target cells) which can be used as a signature in diagnosing and tracking the progression of a particular decease. For example, circulating tumor cells (CTCs) are very rare in individuals without malignancy, but they are present at a wide range of frequencies in patients with various metastatic carcinomas. As a result, the identification of CTCs helps physicians in monitoring and predicting cancer progression. It is also useful to the evaluation of a patient's response to therapy, especially for those patients with metastatic cancer. The number of CTCs in the blood has been shown to correspond to the clinical course of disease and is a predictor of patient's overall survival. In particular, clinical studies have shown correlation between CTC counts and progression of decease for certain types of cancer, such as metastatic breast cancer, colorectal cancer, and prostate cancer. Therefore, much effort has been made in developing methods to capture and isolate biological cells. One of the most commonly used methods involve immunomagnetic cell enrichment using magnetic beads labeled with antibody to capture multiantigent cell and isolate CTCs from blood, tumor tissues, biopsies, or bone marrow samples. Such methods rely on detection of CTCs by binding antibodies to magnetic beads which are mixed into samples and then captured by a fixed magnetic probe covered with a contamination prevention cap. Thereafter, the captured cells are usually cleaned in certain liquid solution to remove unwanted materials such as blood or bone marrow. Then, the cells are released or separated from the contamination prevention cap in order to be collected for cell counts or further study.

The problem with known cell isolation devices using magnetic probe is that in order to change the magnetic field strength at the tip of the probe for the purpose of alternatively capturing and releasing cells enriched with magnetic beads, it either requires alternatively attaching and detaching the contamination prevention cap from the fixed magnetic probe, or requires a complicated automatic control mechanism connected to the probe to move the probe into a position abutting against or separating from the contamination prevention cap. The method of detaching the contamination prevention cap from the magnetic probe severely limits the effectiveness of target cell collection, reduces the sensitivity of cell isolation devices, and as a result hinders the adoption and development of advanced cell isolation techniques. The type of cell isolation device with an automatic control mechanism connected to the probe significantly increases the size, weight, and cost of the probe which, as a result, leads to many other problems, including lower sensitivity in target cell capture and poor device reliability.

Therefore, what is needed is a cell isolation device that does not suffer from the aforementioned problems, a device which handles the cell isolation during cell magnetic incubation without the requirement of physically removing the contamination prevention cap from the magnet probe, a device that does not require a control mechanism connected to the probe in order to move the probe further away or closer to the contamination prevention cap, and at the same time yields a more sensitive cell collection rate.

### BRIEF SUMMARY OF THE INVENTION

The present invention advantageously fills the aforementioned deficiencies by providing a cell isolation device with a floating magnetic probe. This invention simplifies the mechanism of separating the contamination prevention cap from the magnet probe and provides a convenient way in varying the magnetic strength at the tip of the probe in order to capture and release cells. In addition, the present invention produces a consistent magnetic field strength at the tip of the probe during the entire target cell capture process, even when multiple repetitions of the cell capture, release, wash, and collection cycles are required. Furthermore, the present invention enables effective removals of sample material (such as blood, tissues, biopsies, or bone marrow) as well as unwanted magnetic particles attached to the probe. As a result, the present invention brings the benefit of a higher cell capture rate, more consistent cell collection results, and cleaner final collected cell at a lower cost.

The present invention relates to a cell isolation device with a floating magnetic probe, comprising a fixed tube placed in a vertical direction, the tube having with a top cover (or other obstacles inside the fixed tube top portion to serve as a stopper), a position guide rod inside the fixed tube, a magnet head attached to the bottom of the position guide rod, and a contamination prevention cap preferably snugly fit outside the fixed tube at the bottom. The fixed tube can be in any shape, such as circular, rectangular, triangle, or oval, preferably comprising a top portion and a bottom portion, with a wider inner opening for the top portion than that for the bottom portion. The top cover has the same dimension as the top of the fixed tube and tightly fits onto the top of the fixed tube. The position guide rod preferably has a bottom portion with a shape and size that fits loose inside the inner bottom portion of the fixed tube, having a length that is equal to or slightly longer than the bottom portion of the fixed tube. The position guide rod has a top portion that fits loosely inside the top portion of the fixed tube, with a horizontal dimension smaller than the inner opening of the top portion of the fixed tube, but greater than the inner opening of the bottom portion of the fixed tube so that the fixed tube's bottom inner opening serves as a stopper preventing the bottom tip of the position guide rod from moving down beyond the bottom opening of the fixed tube. A magnet head, having a horizontal dimension smaller than the bottom opening of the fixed tube, is attached to the bottom of the position guide rod. The contamination prevention cap preferably has a similar shape as that of the fixed tube, with an inner diameter that is slightly greater than the outside diameter of the fixed tube so that the contamination prevention cap fits onto the outside of the fixed tube. In addition, the tip of the contamination prevention cap preferably has a shape such that the inner magnet head can be in contact with, or at a close proximity to, the tip of contamination prevention cap over a maximum area for the purpose of optimizing the magnetic field at the tip of the contamination prevention cap and efficiently capturing magnetic materials onto its tip.

The inner magnet head is made of magnetic material, either from permanent magnet or other magnetic materials. The position guide rod could be an extension of the magnet head, or may consist of a separate component. This separate component could be made of non-magnetic material or the same material as that of the magnet head. All other components are made of non-magnetic material, including but are not limited to copper, wood, leather, vinyl, canvas, plastics, composites, or glass. Specifically, the contamination prevention cap is made of a non-magnetic material and preferably has a minimal thickness such that the magnetic field generated by the inner magnet head at the outer surface of the contamination prevention cap is not reduced significantly by the cap thickness, while at the same time the cap should not tear or puncture easily during the cell isolation process. The position guide rod helps the magnet head to hold its magnetic field orientation in a predetermined desired direction. The position guide rod and the magnet head are combined together to act as an inner floating magnetic probe inside the fixed tube. The inner floating magnetic probe is freely movable along the fixed tube in the vertical direction and can be moved up and down by various means, such as gravitational or magnetic forces. With the top cover on, the inner floating magnetic probe typically rests at two alternating positions either sitting at the bottom of the contamination prevention cap (“Bottom” position) or abutting against the top cover (“Top” position). As a result, the magnetic field strength at the tip of the contamination prevention cap can be altered between a maximum and a minimum strength without removing the contamination prevention cap itself from the fixed tube.

Without any interference from other forces, the inner floating magnetic probe, due to its gravitational weight, sits at its natural resting state at the bottom of the fixed tube (Bottom position). As a result, the inner magnetic head is in intimate contact with, or at a close proximity to the inner bottom of the contamination prevention cap, creating a strong magnetic field at the tip of the cap and attracting small magnetic objects nearby. This magnetic field pulls magnetic beads in its vicinity, including those magnetic beads that are bound together with the target cells, toward the outer surface of the contamination prevention cap. This force in turn pulls the target cell themselves onto the tip of the contamination prevention cap. When the inner floating magnetic probe is moved upward to its up position abutting against the top cover (Top position), the magnetic field at the contamination prevention cap is reduced significantly, thus making it easier to remove the target cells and magnetic beads from the contamination prevention cap.

During the cell capture process, the fixed tube along with its inner floating magnetic probe and the contamination prevention cap are controlled by a motor as one unit (referred to as the “Probe Unit” hereafter) to move either vertically or horizontally or other predetermined search path to gather target cells distributed in the sample without affecting the magnetic strength at the tip of the contamination prevention cap. After the Probe Unit completes its search path in the sample well, it is placed into a rinse well to remove unwanted non-magnetic materials from the contamination prevention cap. The rinse process can be repeated a few times as needed. Thereafter, the Probe Unit is placed into a target cell collection well.

A repulsive magnetic field from a strong exterior magnet, having an opposite magnetic polarity as that of the inner magnet head, is placed underneath the contamination prevention cap below the cell collection well. Such repulsive magnetic field can be generated and turned on or off by mechanically move an exterior magnet under the cell collection well into or out of the vicinity of the cell collection well. Alternatively, this can be accomplished by turning on or off electromagnetic current which produces a magnetic field with a reverse magnetic polarity as that of the magnet head. The repulsive magnetic field from the exterior magnet pushes the inner floating magnetic probe to move up to rest against the top cover (Top position). Therefore, the magnetic field at the tip of the contamination prevention cap generated by the inner floating magnetic probe is diminished significantly, while the magnetic field generated by the exterior magnet is much stronger. As a result, the strong magnetic force from the exterior magnet pulls the magnet beads and target cells off the contamination prevention cap down to the bottom of the cell collection well. The Probe Unit can then immediately be placed back into the sample well to repeat the previous target cell capture process. The inner floating magnetic probe, due to its gravitational force, drops down automatically to sit in intimate contact with the bottom of the contamination prevention cap. The Probe Unit can be moved by a motor along another predetermined search pattern and pick up any remaining magnetized target cells in the sample container along the way. The above process can be repeated as many times as required without removing the contamination prevention cap, thus making the target cell capture, rinse, and target cell collection cycle efficient, accurate, and economical.

In one particular embodiment of the present invention, the fixed tube, the position guide rod, and the contamination prevention cap are all in cylindrical shapes. The magnet head is in the shape of a ball with its magnetic north and south poles in a perpendicular orientation such that the magnetic field at the center position below the magnet head is substantially vertical. The material for the magnet head is made of Neodymium (Nd). The position guide rod is made of copper. The materials for the fixed tube, the top cover, and the contamination prevention cap are made of hard plastics. The Nd ball has a diameter similar to that of the position guide rod and is tightly attached to the bottom of the position guide rod to form an inner floating magnetic probe. The fixed tube, having its top cap and contamination prevention cap on, with the inner floating magnetic probe resting at the bottom of the fixed tube (or Probe Unit), is placed into a sample well containing CTCs mixed with magnetic beads in the sample. Through a pre-determined movement pattern, the Probe Unit picks up magnetized CTCs and magnetic beads along its path and captures them onto the outside of the contamination prevention cap. The Probe Unit is then put into a rinse well to wash off non-magnetic impurities, and then placed into a cell collection well. A repulsive magnetic field from a strong exterior magnet placed underneath the contamination prevention cap below the cell collection well, having an opposite magnetic polarity as that of the magnet head, is applied to push the inner floating magnetic probe to its Top position. The repulsive magnetic field is generated by an exterior magnet under the cell collection well. The repulsive magnetic field from the exterior magnet pushes the inner floating magnetic probe to move up to rest against the top cover at its Top position. Therefore, the magnetic field at the tip of the contamination prevention cap generated by the inner floating magnetic probe is diminished significantly, while the magnetic field generated by the exterior magnet is much stronger. As a result, the strong magnetic force from the exterior magnet pulls the magnet beads and target cells off the contamination prevention cap down to the bottom of the cell collection well. The Probe Unit can then immediately be placed back into the sample well. The inner floating magnetic probe, due to its gravitational force, drops down automatically to sit in intimate contact with the bottom of the contamination prevention cap. The Probe Unit, as needed, can move along another predetermined movement pattern and pick up any remaining magnetized CTCs in the sample along the way.

This process can be repeated multiple times without removing the contamination prevention cap from the fixed tube to ensure that all the target cells in the sample have been gathered onto the contamination prevention cap and eventually collected by the cell collection well. In this particular embodiment, the present invention advantageously enables, among other things, separating cells during immunomagnetic incubation. In addition, the contamination prevention cap remains closely attached to the permanent magnet when the contamination prevention cap is placed into the cell collection well, with the ability to easily repeat the process as many times as needed. As a result, the cell collection efficiency is significantly improved and thus provides an increased yield in cell collection with a much shorter time required to collect the cells from the sample.

In still another embodiment of the present invention, following the same process as described above, after capturing target cells onto the tip of the contamination probe, the Probe Unit is placed into a sample wash well. A special filter is placed above or on top of the cell wash well. The filter has a grid size such that individual magnetic beads can easily pass through its grid openings but the target cells themselves are too big to pass through. Therefore, as the repulsive exterior magnetic field is turned on, small magnetic beads are pulled through the filter grid openings, and fall onto the bottom of the cell wash well. The target cells, on the other hand, will remain on the top of the filter because the target cells are too big to pass through the filter grid. As needed, the repulsive exterior magnetic field can be turned off, which will cause the inner floating magnetic probe to automatically fall down to be in contact with the contamination prevention cap. The magnetic force from the inner magnet head attracts remaining magnetic material onto the tip of the Probe Unit. This process can be repeated multiple times as needed. After repeated process, the cells remaining on the filter is very pure and free from most of the individual magnetic beads which makes cell counting and identification more accurate and easier to do. In this particular embodiment, the present invention advantageously enables, among other things, a convenient way to separate individual magnetic beads from target cells so that target cells collected are more pure and easier to count or perform subsequent analysis.

In still another embodiment of the present invention, the cell wash well is filled with a liquid, which allows gentle vibration through a mechanism such as ultrasound. These vibrations enable a quick separation between the free magnetic beads attached to the Probe Unit from the target cells. In this particular embodiment, the present invention advantageously enables, among other things, a much more efficient cell separation and purification process.

It is therefore an object of the present invention to provide a cell separation device which captures magnetized target cells into the contamination prevention cap with an inner floating magnetic probe being at its natural bottom position, and then uses a repulsive exterior magnet underneath the cell collection well to push the inner floating magnetic probe inside the fixed tube up and away from the contamination prevention cap to its top position while at the same time pulls the target cells and magnetic beads attached to the contamination prevention cap unto the bottom of the cell collection well, and conveniently repeats such a capture, rinse, collection process as many times as needed during the immunomagnetic incubation to increase the cell collection efficiency.

It is another object of the present invention to provide a cell separation device which easily collects and removes the magnetic beads and cell from the contamination prevention head multiple times without removing the prevention cap from the fixed tube so that the cell collection process can be accomplished in a shorter period of time with a higher collection yield.

It is also an objective of the present invention to provide a cell separation device which conveniently removes free magnetic beads from the target cell by placing a filter above the cell wash well so that the final cells collected are pure and more accurate to count and easier to analyze.

The present invention now will be described more fully hereinafter with reference to the accompanying drawings, which are intended to be read in conjunction with both this summary, the detailed description and any preferred and/or particular embodiments specifically discussed or otherwise disclosed. This invention may, however, be embodied in many different forms and should not be construed as limited to the embodiments set forth herein; rather, these embodiments are provided by way of illustration only and so that this disclosure will be thorough, complete and will fully convey the full scope of the invention to those skilled in the art.

### BRIEF DESCRIPTION OF THE DRAWINGS

The invention may take physical form in certain parts and arrangement of parts, a preferred embodiment of which will be described in detail in this specification and illustrated in the accompanying drawings which form a part hereof and wherein:

FIG. 1 is a view in perspective of a floating magnetic probe including a fixed tube, a position guide rod, and a magnet head attached to the position guide rod.

FIG. 2 is a view in perspective of a cell isolation device in FIG. 1 before and after a repulsive exterior magnet field is turned on beneath a cell collection well.

FIG. 3 is a view taken in perspective of a cell isolation device in FIG. 2 before and after a repulsive exterior magnet field is turned on beneath a cell wash well, with a filter placed above the cell wash well.

FIG. 4 is a view in perspective of a floating magnetic probe including a fixed tube, a magnet head, and a position guide rod, where the position guide rod is an extension of the magnet head.

FIG. 5 is a top view under a microscope of collected cells having most free magnetic micro beads removed.

FIG. 6 is a figure of cell recovery percentage when a single cell or multiple cells are spiked into a human blood sample.

### DETAILED DESCRIPTION OF THE INVENTION

The present invention is directed to a cell isolation device with a floating magnetic probe which enables cell capture and collection during immunomagnetic incubation and provides a low cost, high yield cell collection of cells within a short period of time.

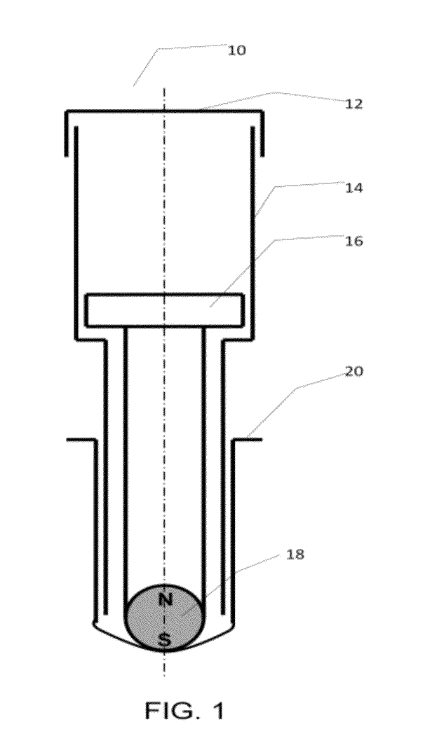

Referring now to the drawings wherein the showings are for purposes of illustrating a preferred embodiment of the invention only and not for purposes of limiting the same, FIG. 1 shows a floating magnetic probe of a cell isolation device, identified in general by the reference numeral 10.

The floating magnetic probe 10 includes a top cover 12 which tightly fits on top of a fixed tube 14, a position guide rod 16 which sits loosely inside the fixed tube 14 with a magnet head 18 attached at the bottom end, and a contamination prevention cap 20 tightly fits to the bottom portion outside the fixed tube 14. The fixed tube 14 is preferably shaped as an elongated cylindrical, with the top half having a wider diameter than that of the bottom half. The position guide rod 16 is also shaped cylindrically with top shaped as a circular plate having a diameter the same as that of the top half of the fixed tube 14. The magnet head 18 is in the shape of a ball, with a diameter the same as the diameter of the body of the position guide rod 16. The contamination prevention cap 20 has a cylindrical shape with a height less than the height of the bottom portion of the fixed tube 14. When no external force is applied, the magnet head 18 together with the position guide rod 16 sits in contact with the contamination prevention cap 20. The magnet head 18 can be made of neodymium alloys (NdFeB), other magnetic materials, or ferromagnetic material magnetized with an electromagnetic field and is attached to the position guide rod 16 with an orientation such that its magnetic north and south polls are positioned at a top or bottom position. The position guide rod 16 could be made of magnetic material or the same material as that of the magnet head 18, or non magnetic material, including, but are not limited to Copper, Aluminum, wood, leather, vinyl, canvas, plastics, composites, or glass.

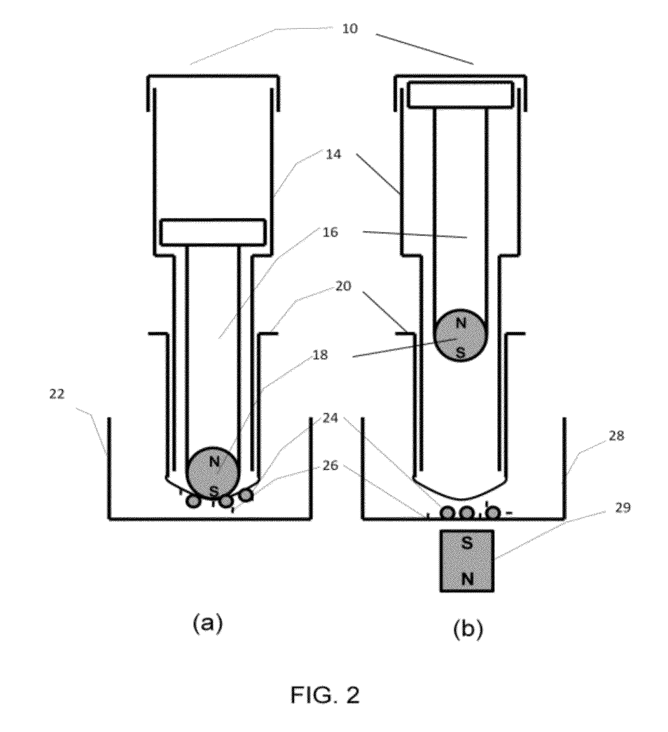

FIG. 2 shows one version of the cell isolation device. After the antibody labeled magnetic beads have been bonded with cells such as CTCs in blood, marrow or other samples, the floating magnetic probe 10 is placed in a sample well 22 as shown in FIG. 2. The floating magnetic probe 10 is placed and moved slowly in the sample well to attract the magnetically bonded target cells 24 (FIG. 2a). At this step, there are no other magnetic fields present except the magnetic field generated by the magnet head 18. The magnet head 18, due to its gravitational weight, stays at the bottom of the fixed tube 14 in direct contact with the contamination prevention cap 20. The magnetic force generated by the magnet head 18 will attract the target cells nearby to the outer surface of the contamination prevention cap 20 because the cells are bonded with one or more small magnetic beads 26. After target cells 24 together with magnetic beads 26 are attracted onto the surface of the contamination prevention cap 20, the cell isolation device 10 is placed into a wash well (not shown in the figures) for cleaning purposes. Thereafter, it is placed into a cell collection well 28 (FIG. 2b). Then, an external opposite magnetic field is applied by an external magnetic source 29. This external magnetic source 29 could be either permanent magnet or electromagnetic. The external magnetic field generated by the external magnetic source 29 is such that it has a reverse magnetic polarization as that of the magnet head 18 and is strong enough to push the magnet head 18 together with the position guide rod 16 up to the upper position in contact with the top cover 12. The cells 24 and the magnetic beads 26 attached to the outside surface of the contamination prevention cap 20 are then pulled away from the contamination prevention cap 20 and dropped into cell collection well 28.

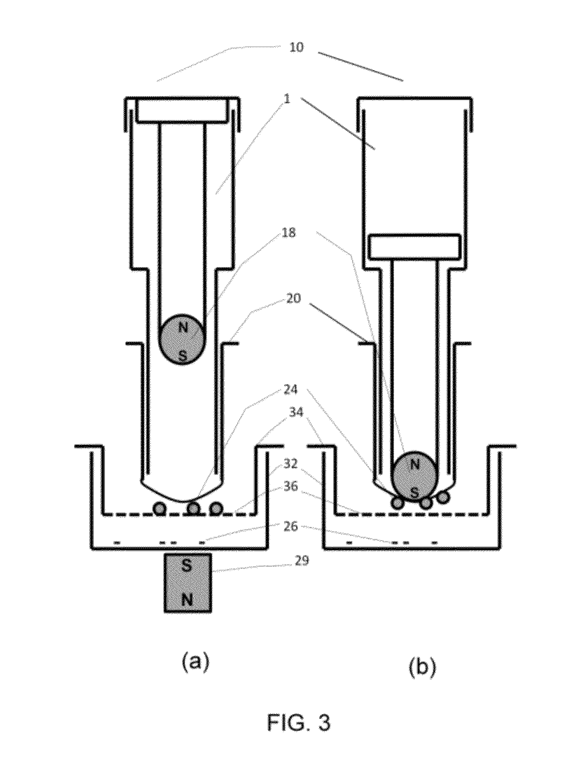

Because the position of the magnet head 18 is mobile and freely floats inside the fixed tube 14, it can easily move together with the contamination prevention cap 20 as a unit from immunomagnetic incubation well to sample cleaning well or rinse well, then to the cell collection well. This significantly decreases the difficulty of keeping constant both the cell attraction magnetic force and the gap between the contamination prevention cap 20 and the bottom of the sample well 22 as well as the bottom of the cell collection well 28, especially when multiple probes are used at the same time. As a result, it improves the cell capture rate and decreases the cell isolation device manufacturing cost. In addition, the freely mobile nature of the magnet head 18 in the vertical direction makes it easier to remove individual magnetic beads 26 from the collected target cell 24 and thus yields very pure cell samples and high cell collection rate at a faster speed. The cell isolation process is further described in FIG. 3.

FIG. 3 represents another version of the cell isolation device. In FIG. 3, after all bonded magnetized cells 24 are attached onto the contamination prevention cap 20, the floating magnetic probe 10 is put into a wash well 32, where an insert filter 34 with membrane filter 36 is placed. An opposite magnet field can be applied from the exterior magnetic source 29 under the wash well 32. When the external magnetic field is turned on, the magnet head 18 is pushed up. The collected cells 24 and the free magnet beads 26 are pulled off from the outer surface of the contamination prevention cap 20. The free magnetic beads 26 will pass through the member filter 36 and drop onto the bottom of the wash well 32 because their sizes are smaller than the size of the micro holes on the member filter 36. The collected cells 24 will stay on top of the membrane filter 36 because their sizes are larger than the size of the micro holds of the filter 36. Thereafter, the external magnetic field from the external magnetic source 29 is turned off. The permanent magnet 18 will fall back to its lower position due to its gravitational weight. The cells 24 on the top of the membrane filter 36 are attached back to the contamination prevention cap 20 by the magnetic force at the bottom of the contamination cap 20 from the magnet head 18. The free magnet beads 26 will stay on the bottom of the wash well 32 because the gap between the membrane filter 36 and the bottom of the wash well 32 weakens the magnetic force from the magnet head 18 applied on them. To achieve better results in removing the free magnet beads 26 from collected cells 24, this procedure may be repeated a few times.

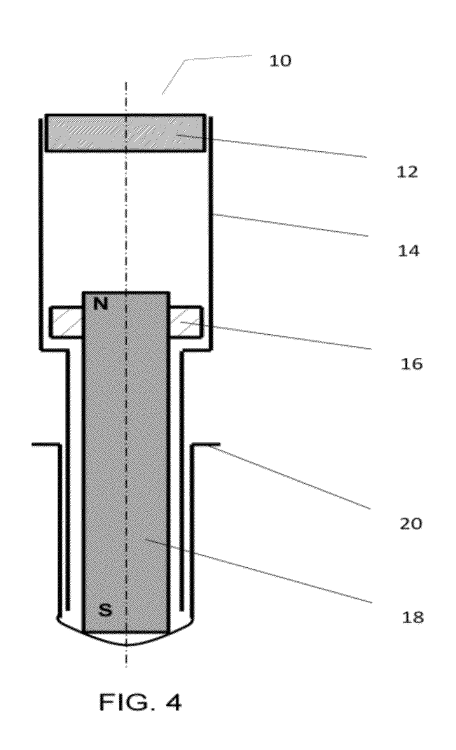

FIG. 4 shows another version of a floating magnetic probe for a cell isolation device, identified in general by the reference numeral 10, which slightly differs from the cell isolation device shown in FIG. 1. The elements are numbered similarly as that in FIG. 1.

The floating magnetic probe 10 includes a top cover 12 which tightly fits side a fixed tube 14, inside the fixed tube 14 sits a position guide rod 16 with its trunk being an extension of a magnet head 18 at the bottom of the rod, and a contamination prevention cap 20 tightly fit to the bottom portion outside the fixed tube 14. The fixed tube 14 is preferably shaped as an elongated cylindrical, with the top half having a wider diameter than that of the bottom half. The position guide rod 16 has a top shaped as a circular plate having a diameter the same as that of the top half of the fixed tube 14. The magnet head 18 also serves as the main trunk of the position guide rod 16 and is in the shape of a cylindrical rod, with a diameter the same as the inner diameter of the bottom half of the fixed tub 14. The contamination prevention cap 20 has a cylindrical shape with a height less than the height of the bottom portion of the fixed tube 14. When no external force is applied, the magnet head 18 together with the position guide rod 16 sits in contact with the contamination prevention cap 20. The magnet head 18 can be made of neodymium alloys (NdFeB), other magnetic materials, or ferromagnetic material magnetized with an electromagnetic field with an orientation such that its magnetic north and south polls are positioned at a top and bottom position respectively. All other parts are made of non-magnetic material, including, but are not limited to Copper, Aluminum, wood, leather, vinyl, canvas, plastics, composites, or glass.

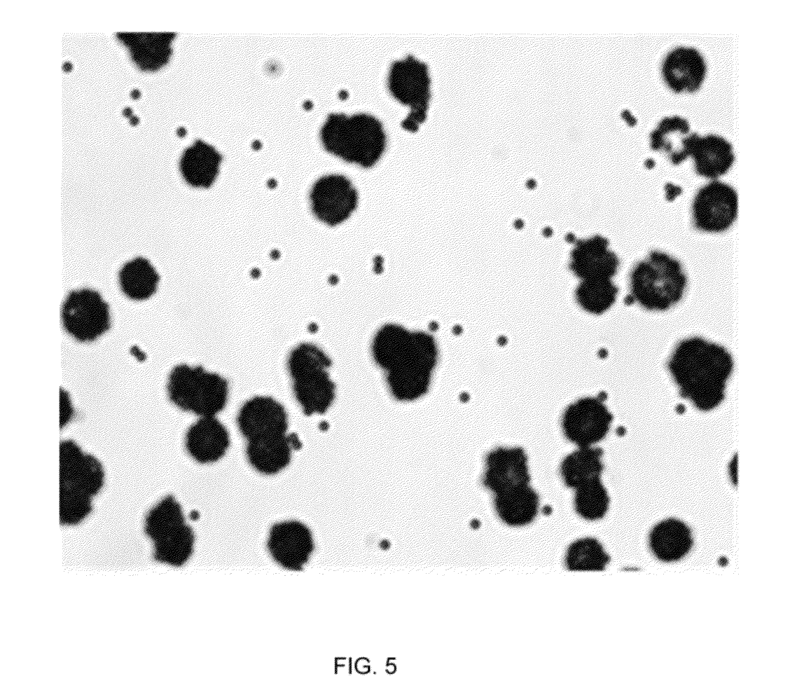

Known cell isolation devices usually capture target cells with a large amount of magnetic micro beads present, which makes it hard to identify target cells under a microscope observation. Present cell isolation device effectively removes micro beads from captured target cells, yielding high purity target cells and increases the resolution of target cell identification and downstream analysis. FIG. 5 shows a top view under a microscope of target cells collected using present invention, free from most micro beads abundantly preset in the sample during immunomagnetic enrichment.

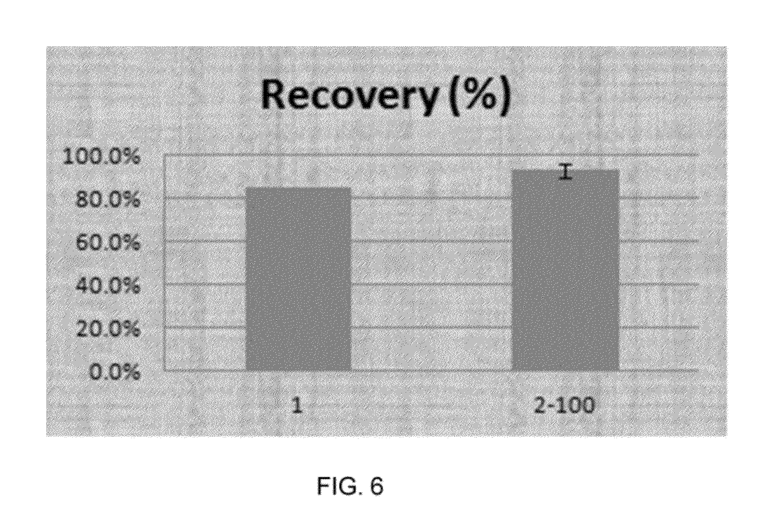

FIG. 6 is a figure of cell recovery rate using present invention when target cells are spiked into a blood sample. When one MCF-7 cell was spiked into a 10 ml human blood sample, an 85% recovery rate for such a single cell was demonstrated using present cell isolation device. When two to one hundred MCF-7 cells were spiked into a 10 ml human blood sample, a greater than 90% recovery rate was achieved in less a than one hour time period.

While the present invention has been described above in terms of specific embodiments, it is to be understood that the invention is not limited to these disclosed embodiments. Many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings.

We claim:

1. A cell isolation device comprising;
a sample container, containing a sample;
a probe adapted to move about inside said sample, which said probe comprises a tube situated vertically having an inner opening wider on the top than that at the bottom, a rod placed inside said tube which said rod having a length shorter than the length of said tube and a protruding portion near the top, an obstacle positioned in the top portion of said tube, a contamination prevention cap attached to the bottom of said tube, a magnet head attached to said rod, said magnet head having its magnetic south and north polls aligned substantially vertically;
a cell collection well, placed near said container; and
an exterior magnetic source, positioned underneath the bottom of said well, wherein a repulsive magnetic field produced by said exterior magnetic source having been adapted to be strong enough to push said magnet head upward to a position pressing against said obstacle when said probe is placed in said well.

2. The cell isolation device as in claim 1, wherein said obstacle is a top cover tightly fit onto the top opening of said tube.

3. The cell isolation device as in claim 1, wherein said obstacle is a thin plate placed inside the top portion of said tube.

4. The cell isolation device as in claim 2, wherein the material of said tube, said top cover, and said cap are made of non-magnetic material.

5. The cell isolation device as in claim 4, wherein the said tube, said top cover, the protruding portion of said rod, and said cap are made of brass, Aluminum, Copper, glass, or plastic, and the remaining portion of said rod and said magnet head are made of Neodymium.

6. The cell isolation device as in claim 5, wherein said tube is shaped as an elongated two-sectional cylindrical tube with the top half having a larger diameter than that of the bottom half, said rod is shaped cylindrically with a diameter the same or slightly smaller than that of the inner diameter of the bottom portion of said tube, with a top shaped as a circular disk with a diameter slight smaller than the inner diameter of the top portion of said tube, and said cap is shaped as an elongated cylinder with a diameter the same or slightly larger than the outer diameter of the bottom portion of said tube and a height equal to or slightly less than the height of the bottom portion of said tube.

7. A cell isolation device as in claim 6, wherein said exterior magnetic source is a permanent magnet with its magnetic north and south polls opposite to that of said magnet head.

8. A cell isolation device as in claim 6, wherein said exterior magnetic source is an electromagnet with an electro-current flowing along a coil in a direction which produces a magnetic field opposite to the direction of said magnetic field produced by the inner magnet head at the tip of said cap.

9. A cell isolation device comprising:
a sample container, containing a sample with one or more cells and magnetic beads;
a probe adapted to move about inside said sample, which said probe comprises a tube situated vertically having an inner opening wider on the top than that at the bottom, a rod placed inside said tube which said rod having a length shorter than the length of said tube and a protruding portion near the top, an obstacle positioned in the top portion of said tube, a contamination prevention cap attached to the bottom of said tube, a magnet head attached to said rod, said magnet head having its magnetic south and north polls aligned substantially vertically;
a wash well, placed near said container, with a membrane filter placed above said well, said filter having micro holes of such a diameter that said magnetic beads in said sample can easily pass through said micro holes while said cells in said sample are too big to pass through; and
an exterior magnetic source, positioned underneath the bottom of said well, wherein a repulsive magnetic field produced by said exterior magnetic source having been adapted to be strong enough to push said magnet head upward to a position pressing against said top cover when said probe is placed in said well, which said repulsive magnetic field can be switched on and off by controlling said exterior magnetic source.

10. The cell isolation device as in claim 9, wherein said exterior magnetic source is made of an exterior permanent magnet with its magnetic north and south polls opposite to that of said magnet head.

11. The cell isolation device as in claim 9, wherein said exterior magnetic source is made of electro-magnetic material with a constant electrical current spiraling around it; and the turning on and off of said repulsive magnetic field is accomplished by turning on and off said constant electrical current.

12. The cell isolation device as in claim 9, wherein the material of said tube, said top cover, and said cap are made of non-magnetic material.

13. The cell isolation device as in claim 9, wherein the said tube, said top cover, said top plate of said rod, and said cap are made of brass, Aluminum, Copper, glass, or plastic, and the remaining portion of said rod and said magnet head is made of Neodymium.

14. The cell isolation device as in claim 13, wherein said tube is shaped as an elongated two-sectional cylindrical tube with the top half having a larger diameter than that of the bottom half, said rod is shaped cylindrically with a diameter the same or slightly smaller than that of the inner diameter of the bottom portion of said tube, with a top shaped as a circular disk with a diameter slight smaller than the inner diameter of the top portion of said tube, and said cap is shaped as an elongated cylinder with a diameter the same or slightly larger than the outer diameter of the bottom portion of said tube and a height equal to or slightly less than the height of the bottom portion of said tube.

15. A cell isolation method comprising moving a tube evenly and slowly inside a sample, said sample containing one or more cells and magnetic beads, wherein a top cover fitting tightly on top of said tube and a contamination prevention cap encasing the bottom portion of said tube, said tube containing a rod with a length shorter than that of said tube with a magnet head attached to the bottom of said rod, said magnetic head having its magnetic south and north polls aligned substantially vertically, said tube attracting said magnetic beads and said cells in said sample onto the outer surface of said cap, and thereafter entering into a collection well, wherein a repulsive magnetic field generated by an external magnetic source under said well pushing said rod up against said top cover, thereby reducing the magnetic field strength by said magnet head at the tip of said cap, thus pulling off said magnetic beads and said cells from the outer surface of said cap onto the bottom of said collection well.

16. The cell isolation method as in claim 15, further comprising, prior to said tube entering into said collection well, placing said tube into a wash well with a membrane filter placed between said cap and said well, wherein a repulsive magnetic field generated by an exterior magnet under said well pushing said rod up against said top cover, thereby reducing the magnetic field strength by said magnet head at the tip of said cap, thus pulling off said objects and said cells from the outer surface of said cap onto said filter, forcing said smaller magnetic beads through said filter and leaving larger said cells on top of said filter, and re-capturing said larger cell resting on top of said filter by turning off said exterior magnet; and repeating such process multiple times when needed.

17. The cell isolation method as in claim 16, wherein said exterior magnet is made of a permanent magnet and the shutting on and off its magnetic fields consists of inserting and removing a magnetic deflecting plate between said exterior magnet and said well.

18. The cell isolation method as in claim 16, wherein said exterior magnet is made of electro-magnetic material with a constant electrical current spiraling around it. The turning on and off the magnetic field of said exterior magnet is accomplished by turning on and off said constant electrical current.
